(19) United States
(12) Patent Application Publication
(10) Pub. No.: US 2007/0212741 A1
(43) Pub. Date: Sep. 13, 2007

(54) Diagnostic markers for sub-acute ruminal acidosis

(76) Inventors: Jan C. Plaizier, Winnipeg (CA); Danis O. Krause, Winnipeg (CA)

Correspondence Address: Fasken Martineau DuMoulin LLP, 2100 - 1075 West Georgia Street, Vancouver, BC, V6E 3G2, US

(21) Appl. No.: 11/372,170

(22) Filed: Mar. 10, 2006

(51) Int. Cl. G01N 33/53

(52) U.S. Cl. 435/7.92

(57) ABSTRACT

A diagnostic method and kits for detecting sub-acute ruminal acidosis in livestock animals. The method comprises collecting a blood sample from a livestock animal, contacting the blood sample with a kit provided with at least one marker derived from at least one acute phase protein associated with inflammatory responses. The marker is an antibody configured to react with said acute phase protein. The antibody reacts with the acute phase protein present in the blood sample as a consequence of an inflammatory response to sub-acute ruminal acidosis thereby providing a detectable and preferably, a quantifiable result. The marker comprises at least one antibody configured to react with serum amyloid-A or haptoglobin. The diagnostic kit comprises a solid support structure provided with at least one marker configured to react with an acute phase protein. The diagnostic kit may be an ELISA kit Alternatively, the diagnostic kit may be a paper strip.

### FIELD OF THE INVENTION

This invention relates to diagnostic markers. More particularly, this invention relates to diagnostic markers for detecting sub-acute ruminal acidosis in livestock.

### BACKGROUND OF THE INVENTION

Carbohydrates are the primary energy source for livestock and typically contribute 70% to 80% of livestock dry matter feed while protein, fats and minerals make up the remainder. Ruminant livestock have specialized digestive systems and processes which extract maximal nutritive value from these inputs. Cattle feed comprises two main carbohydrate categories which are (a) soluble components in the forms of sugars and starch, and (b) fibre components from cell walls in the forms of cellulose and hemicelluloses, lignins, and pectins. Sugars, starch and fibre are converted through fermentation by rumen-dwelling bacteria into volatile fatty acids which serve as the main sources of energy for livestock metabolism, growth and production. The primary volatile acids produced by rumen bacteria are acetate (55%-70%), propionate (15% to 30%), and butyrate (5% to 15%). An acetate:propionate ratio of about 2.4:1 is considered optimal for physiologically efficient rumen fermentation. It is known that starch/sugar-digesting rumen bacterial metabolism is optimal in the pH range of 5.5 to 6, while fibre-digesting rumen bacterial metabolism is optimal in the pH range of 6.0 to 6.8. Therefore, it is essential to manage the feed supply to high-production livestock so that the rumen pH is maintained at about 6.0.

The need to increase beef production and milk production has resulted in the development and use of intensive feeding strategies for feedlot and dairy cattle that are based on precise manipulation of the fibre-to-concentrate ratio of carbohydrate sources, e.g., coarse hay, chopped hay, silage, whole grains, and ground grains, provided to the livestock. Those skilled in these arts refer to hay and silage as fibre components and to whole and/or crushed and/or ground grain as the concentrate components. It is known that increasing the grain i.e., concentrate component of feed which is rich in rapidly fermentable sugars and starch, will provide short-term increases in weight gains and milk production. However, supplying increasing amounts of grain concentrate relative to the fibre component significantly increases the metabolic activity of sugar/starch-fermenting rumen bacteria relative to the fibre-digesting bacteria resulting in increasing acidification of the rumen fluids.

Sub-acute ruminal acidosis (SARA) was defined by Cooper et al (1996, Update on Rumensin/Tylan/Micoryl for the Professional Feedlot Consultant, Elanco Animal Health, Greenfield, Ihd., USA) as a metabolic disorder that is characterized by episodes of low rumen pH in the range of 5.2 to 5.6. SARA is known as a major physiological problem for beef cattle during adaptation to high grain finishing diets in feed lot operations, and in dairy cows especially during the transition period after calving. The clinical signs of SARA are variable and therefore, it is very difficult to identify and diagnose animals suffering from the SARA disorder. Some indirect signs of SARA-affected livestock include erratic feeding intake, anorexia, intermittent diarrhea, dehydration, unexplained abscesses and laminitis (Nocek, 1997, J. Dairy Sci. 80: 1005-1028; Kleen et al., 2003, J. Vet. Med. A 50: 406-414). These symptoms are not exclusively specific to SARA and this often leads to SARA being dismissed as other problems such as poor forage quality or poor freed management. Another significant problem is the absence of diagnostic tests for detection of SARA causes reliance on subjective recognition of the syndrome of the secondary clinical signs mentioned above within a herd (Nordlund, 2003, Preconvention Seminar 7, 36^{th} Annual Conference, American Association of Bovine Practitioners). A tentative diagnosis of SARA is confirmed by herd response to corrective nutrition by adjustments to the fibre-to-concentrate ratios of the feed.

It has been proposed that collection and analyses of rumen fluid could be useful if diagnosing SARA, particularly in conjunction clinical observations (Garrett et al., 1999, J. Dairy Sci. 82: 1170-1178; Nordlund, 2003). U.S. Pat. No. 6,694,161 discloses an invasive device for indwelling installation through the side of an animal and related methods for in vivo measurements of rumen pH. U.S. Pat. No. 6,624,688 discloses portable equipment for collecting ruminal fluids and related methods for analysis and use of the fluids.

### SUMMARY OF THE INVENTION

The exemplary embodiments of the present invention, at least in preferred forms, are directed to diagnostic methods and kits for detecting sub-acute ruminal acidosis.

According to a preferred embodiment of the present invention, there is provided a method for detecting sub-acute ruminal acidosis in livestock animals by contacting a blood sample collected from a livestock animal, with a diagnostic device provided with at least one marker configured to react with a metabolite produced by the animal in direct response to sub-acute ruminal acidosis. It is preferred that the blood sample is a peripheral blood sample.

According to one aspect, the marker is preferably derived from a selected acute phase protein associated with an inflammatory response within said livestock animals. The marker is configured to react with the selected acute phase protein present in the blood sample. In a preferred form, the marker is an antibody configured to react with the selected acute phase protein. It is preferred that the acute phase protein is selected from the group comprising serum amyloid-A and haptoglobin.

According to another aspect, there is provided a plurality of markers derived from at least two acute phase proteins associated with an inflammatory response within said livestock animals. In a preferred form, the plurality of markets comprises at least one antibody configured to react with serum amyloid-A and at least one antibody configured to react with haptoglobin.

According to another aspect, the blood sample is separated into at least a serum portion and a plasma portion. The serum portion is contacted with a first diagnostic device provided with at least one marker configured to react with haptoglobin. It is preferred that the marker is an antibody configured to react with haptoglobin. The plasma portion is contacted with a second diagnostic device provided with at least one marker configured to react with serum amyloid-A. It is preferred that the marker is an antibody configured to react with serum amyloid-A.

According to another preferred embodiment of the present invention, there is provided a diagnostic kit for detecting acute phase proteins produced by livestock animals experiencing sub-acute ruminal acidosis. The diagnostic kit comprises a solid support structure provided with at least one marker selected for reacting with an acute phase protein produced by livestock animals in response to sub-acute ruminal acidosis. It is preferred that the marker is engaged with and cooperates with the solid structure.

According to one aspect, the marker is preferably an antibody configured to react with a selected acute phase protein associated with an inflammation response. It is preferred that the acute phase protein is selected from the group comprising serum amyloid-A and haptoglobin.

According to another aspect, there is provided a plurality of markers derived from at least two acute phase proteins associated with an inflammatory response within said livestock animals. In a preferred form, the plurality of markers comprises at least one antibody configured to react with serum amyloid-A and at least one antibody configured to react with haptoglobin.

According to yet another aspect, the diagnostic kit is contactable and reactable with a blood sample collected from a livestock animal. It is preferred that the diagnostic kit is contactable and reactable with a serum portion and a plasma portion of a blood sample. In a preferred form, the reaction between the diagnostic kit and the blood sample and/or the serum portion and/or the plasma portion containing therein at least one acute phase protein associated with an inflammatory response, is quantifiable.

In a preferred form, the diagnostic kit of the present invention comprises an ELISA test kit.

In another preferred form, the diagnostic kit of the present invention comprises a paper strip.

### BRIEF DESCRIPTION OF THE DRAWINGS

The present invention will be described in conjunction with reference to the following drawings, in which:

FIG. 1 is a graph showing the effects of imposed SARA on: (a) ruminal concentrations of lypopolysaccharides (LPS) (log₁₀ Eu mL⁻¹), (b) peripheral blood concentrations of haptoglobin (Hp) (mg mL⁻¹), and (c) peripheral blood concentrations of serum amyloid-A (SAA) (μg mL⁻¹);

FIG. 2 is a graph showing the effects of diet on changes in the total volatile fatty acid concentrations (VFA) (mM mL⁻¹) in rumen fluid over time (bar graph). The average changes in rumen fluid pH over time are shown as a line graph. Bars indicate SE;

FIG. 3 is a graph showing effects of diet on changes in the concentration of ruminal LPS (log₁₀ Eu mL⁻¹). Bars indicate SE.

FIG. 4 is a graph showing the effects of diet on diurnal changes in ruminal LPS concentrations (log₁₀ Eu mL⁻¹) (bar graph). Mean rumen pH (line graph) is shown for the same time period. Bars indicate SE;

FIG. 5 is a graph showing the effects of diet on total coliform counts (TCC) (log₁₀ cfu mL⁻¹) in rumen fluid of steers. Bars indicate SE;

FIG. 6 is a graph showing is a graph showing total dry matter intake (DMI) (kg d⁻¹) for dairy cows fed: (a) a control total mixed rations (TMR) diet only (▪), or (b) a diet comprising total mixed rations plus wheat-barley concentrate to induce SARA (SARA diet) (□). Each bar represents mean ±SEM total dry matter intake (n=8). *=P<0.05, Control vs. SARA;

FIG. 7 is a graph showing effects of diet on ruminal LPS levels in 4 cows.
Week 1: all cows fed TMR,
Week 2: cows 1 and 2 were fed SARA diet, while cows 3 and 4 were fed TMR control,
Week 3: cows 1 and 2 were fed TMR, while cows 3 and 4 were fed the SARA diet,
Week 4: all cows were fed the SARA diet;

FIG. 8 is a graph showing ruminal LPS levels in dairy cows fed: (a) a TMR diet (), or (b) the SARA diet (o). Each bar represents mean LPS concentration in log₁₀ Eu mL⁻¹±SEM (n=8). **=P<0.01, Control vs. SARA; ***=P<0.001, Control vs. SARA in samples collected at 3:00 pm every day; and

FIG. 9 is a graph showing total coliform counts (TCC) (log₁₀ cfu mL⁻¹) of cows fed: (a) a TMR diet (▪), or (b) the SARA diet (□), enumerated in rumen fluid at 6-hr intervals over 2 days and averaged across four periods. Each bar represents mean ±SEM TCC in log₁₀ cfu mL⁻¹ (n=16). *=P<0.05, Control vs. SARA.

### DETAILED DESCRIPTION OF THE INVENTION

Exemplary embodiments of the present invention as disclosed herein provide diagnostic methods and kits for detecting sub-acute ruminal acidosis in livestock animals.

Stress encounters such as tissue injury or trauma, infection, and neoplastic or immunological disorders, stimulate a systematic series of immunological and inflammation reactions in mammalian systems which are typified by the synthesis and release into the bloodstream of pro-inflammatory cytokines which serve to activate the vascular system and inflammatory cells to produce more cytokines and other inflammatory mediators which circulate throughout the body in the blood (Gruys et al., 2005, J. Zhejiang Univ. Sci. 6B(11): 1045-1056). This series of reactions is commonly referred to as the “acute phase response” and is characterized by the appearance and accumulation in the vascular systems of certain specific proteins commonly referred to as “acute phase proteins”. Serum amyloid-A and haptoglobin are two acute phase proteins that are known inflammatory markers in cattle for responses to stresses such as tissue damage, bacterial infections, and injections of lipopolysaccharides (Connor et al., 1988, Res. Vet Sci. 44: 82-88; Am. J. Vet Res. 50: 1690-1694; Alsemgeest et al., 1994, Vet. Q. 16: 21-23; Deignam et al., 2000, Res. Vet Sci. 69: 153-158; Jacobsen et al., 2004).

Lipopolysaccharides are the major constituents of the cell walls of Gram-negative bacteria, consisting of a lipid part and two polysaccharide parts. Lipopolysaccharides are highly immunogenic, and when elevated levels occur in mammalian systems as a consequence of microbial infections, they stimulate the production of pro-inflammatory cytokines pyrogen interleukin-1 and tumor necrosis factor during the initial stages of the acute phase response as precursors to synthesis of acute phase proteins. However, ruminant livestock animals are continually exposed to and adapted to relatively low levels of lipopolysaccharides in the rumen fluid since rumen microbial populations primarily comprise Gram-negative bacteria which shed outer membrane fragments containing lipopolysaccharides during their normal growth, metabolism and death.

We have discovered that imposing SARA on livestock animals through manipulation of their dies results in significantly elevated levels of lipopolysaccharides in their rumen fluid within hours of their experiencing the SARA conditions, and that the elevated lipopolysaccharide levels are maintained over several diurnal cycles. We have surprisingly discovered that at least one acute phase protein, i.e., serum amyloid-A is detectable in peripheral blood samples collected from livestock animals within 24 hours of the occurrence of SARA and elevated lipopolysaccharide conditions in their rumens, and that the levels of serum amyloid-A tend to increase with the duration of SARA. We have further surprisingly discovered that a second acute phase protein, i.e., haptoglobin is detectable in peripheral blood samples collected from livestock animals within 72 hours of the occurrence of SARA and elevated lipopolysaccharide conditions in their rumens. Therefore, the present invention disclosed herein provides a method for diagnosing SARA in individual livestock animals wherein a blood sample is collected from an animal and tested for the presence of acute phase proteins serum amyloid-A and/or haptoglobin. The blood sample may be a peripheral blood sample and is preferably separated into a serum portion and a plasma portion. The blood sample or its separated portions, is contacted with a test kit provided with at least one marker configured to react with either serum amyloid-A or haptoglobin. The marker configured to react with serum amyloid-A is preferably an antibody reactable with the serum amyloid-A protein while the marker configured to react with globulin is preferably an antibody reactable with the globulin protein Those skilled in these arts will understand that antibodies reactive with serum amyloid-A proteins or, alternatively, globulin proteins may be produced by following standard laboratory practices such as those disclosed by Howard et al. (2001, Basic Methods in Antibody Production and Characterization, CRC Press, the disclosure of which is incorporated herein by reference). Alternatively, commercial produced antibodies specific to serum amyloid-A proteins or haptoglobin proteins, such as those available from Fitzgerald Industries International Inc. (Concord, Mass., USA) and Acris Antibodies Gmbh (Hiddenhausen, Germany), may be used in the methods of the present invention. A blood sample collected from an animal experiencing the early stages of SARA will contain detectable levels of serum amyloid-A in its plasma portion, while a blood sample collected from an animal that has experienced SARA for an extended period (i.e., several days) will contain elevated detectable levels of serum amyloid-A in its plasma portion and detectable levels of haptoglobin in its serum portion. It is within the scope of this invention to provide reference levels of serum amyloid-A and haptoglobin in non-SARA-stressed animals for comparison with levels of these acute phase proteins in SARA-stressed animals and to assign quantifiable values to the severity of SARA stresses. The present invention also provides kits configured to detect acute phase proteins in blood samples and portions thereof, collected from livestock animals. The kits comprise at least one marker configured to react with a selected acute phase protein present in a blood sample or portions thereof. The marker is preferably an antibody configured to react with serum amyloid-A or haptoglobin. If so desired, the kits may be provided with an antibody configured to react with serum amyloid-A and an antibody configured to react with haptoglobin present in a blood sample or portions thereof. The kits are preferably provided with a solid support structure for engaging thereon and cooperating therewith the markers and antibodies of the present invention. The kits of the present invention may comprise ELISA test kits. Alternatively, the kits may comprise paper strips. It is within the scope of this invention to provide kits configured to provide quantifiable values for the levels of serum amyloid-A and/or haptoglobin present in a blood sample or portions thereof, based on correlating color intensities of reactions produced by the quantities of serum amyloid-A markers reacting with serum amyloid-A proteins, and to the quantities of haptoglobin markers reacting haptoglobin proteins.

The diagnostic methods and kits of the present invention for detecting sub-acute ruminal acidosis in livestock animals are described in more detail in the following examples which are intended to be exemplary of the invention and are not intended to be limiting.

### EXAMPLE 1

Three adult ruminally fistulated Jersey steers were kept in metabolism crates at the Animals Science Research Unit at the University of Manitoba (Winnipeg, MB, Canada) throughout the experiment, in accordance with the guidelines of the Canadian Council of Animal Care. The experimental design was a 3×3 Latin square with 21-day periods that were divided into a 5-day treatment period and 16-day rest period. During the treatment period, SARA was induced in the steers by offering diets 1, 2 and 3. Diets were combinations of a pelleted wheat-barley concentrate and chopped alfalfa bay in the following ratios (as fed basis): diet 1 (4 kg:6 kg); diet 2 (5 kg:5 kg); diet 3 (6 kg:4 kg). The concentrate consisted of 50% wheat and 50% barley. Wheat-barley concentrate and hay were offered in separate meals. All animals were offered 1 kg of chopped alfalfa hay at 9:00 am, followed by two thirds of their allocation of concentrate at 11:00 am. The remainder of the concentrate was offered at 1:00 pm. At 5:00 pm, concentrate not eaten was removed and the steers were offered chopped alfalfa hay, which they had access to throughout the night. The steers were fed hay ad libitum during the 16-day rest period between treatments. The last two days of the rest period when only hay was fed were designated D-2 and D-1. Days when concentrate was fed were designated D 1 to D 5. Dry matter (DM), crude protein (CP) and neutral detergent fibre (NDF) contents were 93.0%, 18.9%, 27.3% for the wheat-barley concentrate and 84.8%, 14.9% and 45.7% for chopped alfalfa hay respectively.

Rumen pH was measured continuously throughout the 5-day treatment period using indwelling pH probes as described by Cumby et al. (2001, Can. J. Anim. Sci. 81: 149-152). Measurements were made every second and were averaged over 60 sec. Rumen fluid pH data were summarized as average pH, time in minutes below pH 6.0 and time in minutes below pH 5.6, area (time×pH) below pH 6.0, and area (time×pH) below pH 5.6 for each 24-h period. Rumen fluid samples were collected into sterile plastic tubes from the ventral sac of the rumen at 9:00 am and 2:00 pm every day during the 5-day treatment period. Each sample was mixed thoroughly after which, a 25-mL sub-sample was transferred into a sterilized centrifuge tube and centrifuged for 30 minutes at 10,000 xg. The supernatant was passed through a disposable 0.22 μm sterile, pyrogen free filter (Millex®, Millipore Corporation, Bedford, Mass., USA). Samples were then heated at 100° C. for 30 minutes before being stored at −20° C. for determination of rumen LPS using the Limulus amebocyte lysate (LAL) assay following the method described by Levin et al. (1964, Bull. John Hopkins Hosp. 115: 337). The assay was performed using a 96-well microplate (BioWhittaker Inc. Walkersville, Md., USA) with absorbance read at 405 nm using a microplate reader (Bio-Rad®, Model 3550, Bio-Rad Laboratories Inc., Hercules, Calif., USA). Samples were diluted 1,000-fold using pyrogen-free water to determine the non-inhibitory dilution with the final dilution being made of 50% diluted sample and 50% β-G-blocker (Bio Whittaker kit catalogue number N190; component number: B50-700). β-G-blocker blocks the reactivity of LAL to β-1,3-glucan conferring increased endotoxin specificity to the LAL test Non-inhibitory dilution is achieved when 75±25% of spike is recovered in positive control samples. An average recovery rate of 78.0±8.6% (n=8) was achieved in positive controls with a sample dilution of 1:60,000.

Two blood samples (7 mL) were collected by tail venipucture from each steer on days −2, −1, 1, 2, 3, 4, and 5, into serum and plasma tubes at 9:00 am each day. Serum and plasma were harvested by centrifuging samples at 3,000 rpm for 30 minutes. Haptoglobin and serum amyloid-A were determined in serum and plasma, respectively, using ELISA Tridelta® Phase range assay kits (Tridelta Diagnostics Inc., Morris Plains, N.J., USA; catalogue numbers TP-801 and TP-802, respectively). For haptoglobin, serum samples were diluted 1:5 with phosphate buffered saline, then mixed by vortexing after which, 7.5 μL of each sample were added to duplicate wells of a 96 well microtiter plate. Stabilized hemoglobin diluted 1:1 with hemoglobin diluent, was added (100 μL) to the diluted sample. Chromogen and substrate mixed in a ratio of 9:5 (140 μL), were then added to the reaction mixture and incubated at room temperature for five minutes. Absorbance was read immediately at 630 nm using a microplate spectrophotometer (Spectra Max® 340 PC, Molecular Devices Corporation, Sunnyvale, Calif., USA).

For serum amyloid-A, plasma samples were diluted 1:500 in diluent buffer, then mixed by vortexing after which, 50 μL of each sample were added to each well of a 96 well plate coated with 50 μL of biotinylated anti-serum amyloid-A monoclonal antibody (diluted 1:100 in 1x diluent buffer). The microtiter plate was covered and incubated at 37° C. for at least 1 h, and then washed four times with diluted wash buffer to remove unbound material. Streptavidin horseradish peroxidase conjugate was diluted 1:4,000 in diluent buffer and 100 μL were added to each well. The plate was incubated at room temperature in darkness for 30 minutes. The microtiter plate was washed again as before and tapped dry. Substrate (100 μL) was added to the plate, which was then incubated at room temperature in darkness for an additional 30 minutes. Stop solution was added, and the plate was read in a micro plate reader (Bio-Rad® Model 3550, Bio-Rad laboratories Inc., Hercules, Calif., USA) at 450 nm.

Data were analyzed using mixed model analysis with the first order autoregressive covariance structure using SAS/STAT® software (SAS Institute Inc, Cary, N.C., USA). The following model was used: Yᵢⱼₖₗ=μ+αᵢ+βⱼ+Dₖ+(α×β)ᵢⱼ+Tᵢ+(D×T)ₖₗ+eᵢⱼₖₗ where Yᵢⱼₖₗ=observations for dependent variables, μ=overall mean; αᵢ=average effect of period i; βⱼ=average effect of the animal j; Dₖ=average effect of dietary treatment; (α×β)ᵢⱼ=animal×period interaction which was the main plot error; Tᵢ=average effect of time in days for feed intake, or hours since the beginning of feeding concentrate for blood metabolites or endotoxin concentration; (D×T)ₖₗ=interaction between time and dietary treatment; and eᵢⱼₖₗ=subplot error. LPS concentrations were log-transformed to correct non-homogenous residual errors. Orthogonal contrasts were used to compare baptoglobin, serum amyloid-A and log-transformed LPS concentrations during the period when only hay was fed with the period when the SARA diet was feed. Mean differences for rumen pH data and dry matter intake were separated using Tukey's multiple comparison procedure.

The data in Table 1 show that the average rumen fluid pH, time and area with pH below 6.0 or 5.6 did not differ among the two diets. However, adding the wheat-barley concentrate to the total mixed rations decreased average daily rumen pH from d 1 to a nadir on d 4. Both time below pH 6.0 and time below pH 5.6 increased from d 1 to a peak on d 4. The area below pH 6.0 also increased from d 1 to a peak on d 4 (Table 1). It was clear that SARA was successfully induced on these days because the test steers spent 187 and 174 minutes per day with pH below 5.6 on days 4 and 5, respectively.

Offering different amounts of concentrate and hay was meant to induce SARA to differing extents. Thus, the treatments were designed to cause significantly differences in the dry matter intake of the test animals. Intake of both concentrate and hay was variable in all the treatments (Table 2). However, offering 6 kg of concentrate resulted in more variable intake response compared to the other two diets. Dry matter intake of hay was more variable than that of concentrate and may be the reason for the treatment by day interaction (Table 2). The test animals went through cycles in which a high intake on one day was followed by low intake the following day (Table 1). Averaged across treatments, the decrease in dry matter intake was greatest between d 3 and d 4 for the wheat-barley concentrate, and between d 2 and d 3 for the hay. The steers appeared to reduce hay intake in favor of the concentrate on d 2 and d 3.

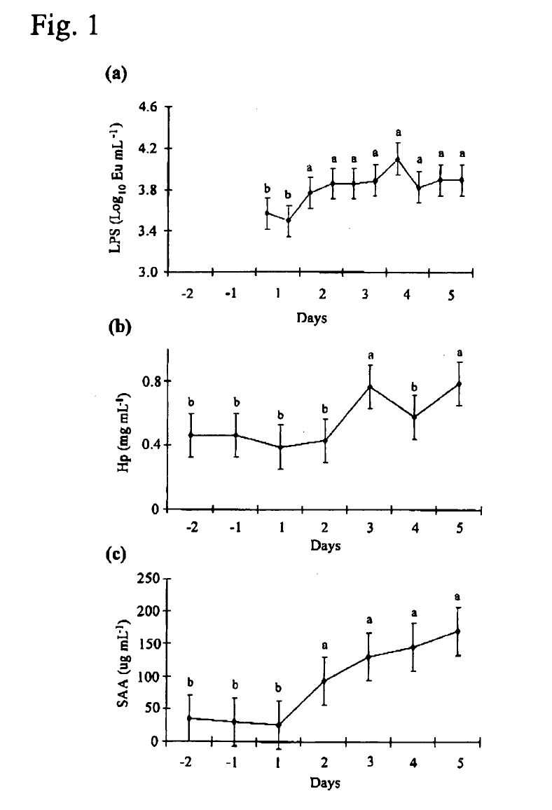

Different levels of wheat-barley concentrate in the diets did not affect LPS concentration. However, compared to when only hay was fed, wheat-barley concentrate increased LPS concentration from d 1 to a peak on d 4 (P<0.05) (FIG. 1a). The peak coincided with the day when the time with pH below 5.6 was greatest. Ruminal LPS increased from 3,715 Eu mL⁻¹ before concentrate was fed to peak on d 4 at 12,589 Eu mL⁻¹. This may have been due to an increase in free LPS under acidic conditions. High concentrations of LPS with low rumen fluid

TABLE 1
Rumen pH variables of steers offered different levels of wheat-barley concentrate and
chopped alfalfa hay to induce sub-acute ruminal acidosis.

| | Diet¹ | | | | Day | | | | | | Effect, P value | | |
|---|---|---|---|---|---|---|---|---|---|---|---|---|---|
| | Diet 1 | Diet 2 | Diet 3 | SEM | 1 | 2 | 3 | 4 | 5 | SEM | Diet | Day | Diet × Day |
| Average pH | 6.39 | 6.29 | 6.26 | 0.05 | 6.49^{c} | 6.20^{cd} | 6.27^{c} | 6.19^{d} | 6.24^{cd} | 0.10 | 0.19 | 0.006 | 0.057 |
| Time < pH 5.6 min/d | 42^{b} | 117^{ab} | 134^{a} | 33.1 | 5^{d} | 80^{cd} | 78^{cd} | 187^{c} | 174^{c} | 42.3 | 0.045 | 0.013 | 0.11 |
| Time < pH 6.0 min/d | 308 | 369 | 404 | 77.3 | 9^{d} | 471^{c} | 425^{c} | 589^{c} | 480^{c} | 88.0 | 0.64 | <0.001 | 0.14 |
| Area < pH 5.6, min × | 3 | 25 | 35 | 13.6 | 1 | 7 | 10 | 54 | 49 | 18.1 | 0.17 | 0.096 | 0.19 |
| pH/d | | | | | | | | | | | | | |
| Area < pH 6.0, min × | 71 | 124 | 125 | 28.9 | 2^{d} | 104^{cd} | 111^{cd} | 189^{c} | 170^{c} | 37.5 | 0.20 | 0.005 | 0.13 |
| pH/d | | | | | | | | | | | | | |

¹Diet 1 = 4 kg of wheat-barley concentrate and 6 kg of chopped alfalfa hay offered daily; Diet 2 = 5 kg of wheat-barley concentrate and 5 kg of chopped alfalfa hay offered daily; Diet 3 = 6 kg of wheat-barley concentrate and 4 kg of chopped alfalfa hay offered daily.
^{a,b}LS means for diets with different letter superscripts within a row were different (P < 0.05).
^{c,d}LS means for days with different letter superscripts within a row were different (P < 0.05).

TABLE 2
Feed intake of steers offered different levels of wheat-barley concentrate
and chopped alfalfa hay to induce subacute ruminal acidosis.¹

| | Day | | | | | | Effect, P value | | |
|---|---|---|---|---|---|---|---|---|---|
| Dry matter intake | 1 | 2 | 3 | 4 | 5 | SEM | Treatment | Day | Treatment × Day |
| Concentrate (kg of DM/d) | | | | | | | | | |
| Diet 1 | 3.72 | 2.68 | 3.14 | 2.62 | 2.84 | 0.75 | | | |
| Diet 2 | 4.65 | 4.65 | 4.05 | 3.26 | 3.45 | 0.75 | 0.005 | 0.01 | 0.28 |
| Diet 3 | 5.61^{a} | 4.47^{a} | 5.61^{a} | 4.26^{a} | 1.19^{b} | 0.75 | | | |
| Diet means | 4.66^{a} | 3.93^{a} | 4.27^{a} | 3.38^{ab} | 2.73^{ab} | 0.56 | | | |
| Chopped alfalfa hay (kg d⁻¹) | | | | | | | | | |
| Diet 1 | 5.06^{a} | 4.69^{ab} | 2.59^{b} | 3.71^{ab} | 3.50^{ab} | 0.48 | | | |
| Diet 2 | 4.24^{a} | 4.40^{a} | 4.08^{a} | 2.78^{b} | 2.08^{b} | 0.48 | 0.085 | <0.0001 | 0.036 |
| Diet 3 | 3.72^{a} | 4.25^{a} | 3.35^{ab} | 1.98^{ab} | 0.83^{b} | 0.48 | | | |
| Diet means | 4.34^{a} | 4.45^{a} | 3.34^{b} | 2.82^{bc} | 2.14^{c} | 0.28 | | | |

¹Diet 1 = 4 kg of wheat-barley concentrate and 6 kg of chopped alfalfa hay offered daily; Diet 2 = 5 kg of wheat-barley concentrate and 5 kg of chopped alfalfa hay offered daily; Diet 3 = 6 kg of wheat-barley concentrate and 4 kg of chopped alfalfa hay offered daily.
^{a,b,c}Means with different letter superscripts within each row were different (P < 0.05).

pH may be due to increased lysis of dead bacterial cells and or shedding of free LPS from rapidly growing gram-negative bacteria.

Animals fed the different diets did not give evidence of significantly different haptoglobin concentrations in their blood serum. However inducing SARA increased concentrations of haptoglobin from 0.43±0.14 mg mL⁻¹ when only hay was fed to 0.79±0.14 mg mL⁻¹ on d 5 of the treatment period (P<0.05) (FIG. 1b). Inducing SARA resulted in higher haptoglobin concentrations on d 3 and d 5 (P<0.05).

Serum amyloid-A concentrations were not significantly different among the different diets but the concentrations in blood plasma increased from 33.6±36.53 μg mL⁻¹ when only hay was fed to 170.7±36.53 μg mL⁻¹ on d 5 (P<0.001) compared to when concentrate-amended total mixed rations was offered in addition to the hay (FIG. 1c).

In summary, offering steers wheat-barley concentrate mixed with chopped alfalfa hay successfully induced SARA, particularly on d 4 and d 5 of this study while dry matter intake declined from d 1 to d 5. Inducing SARA increased the concentration of LPS in rumen fluid, plasma serum amyloid-A, and serum haptoglobin concentrations. The acute phase protein profiles, i.e., serum amyloid-A and haptoglobin, indicate that as the time with pH below 5.6 increased, the intensity of the acute phase response increased. While serum amyloid-A concentrations increased within 24 hours after the first feeding of the wheat-barley concentrate, the haptoglobin concentrations did not change until the third day of feeding the concentrate-amended total mixed rations thereby providing an indication that the duration and severity of sub-acute ruminal acidosis may be assessed by monitoring the appearance of and quantifying these individual markers. Inducing SARA increased rumen LPS concentration and initiated an acute phase response, i.e., creased the levels of serum amyloid-A and haptoglobin in peripheral blood.

### EXAMPLE 2

Three adult ruminally fistulated Jersey steers were gradually adapted to a pelleted wheat-barley concentrate by feeding graded levels of the concentrate and chopped alfalfa hay in an experimental design fashioned after Cooper and KJopfenstein (1996, Update on Rumensin/Tylan/Micotyl for the Professional Feedlot Consultant, Elanco Animal Health, Greenfield, Ill., USA). The steers were kept in metabolism crates housed in the Animal Science Research Unit building at the University of Manitoba (Winnipeg, MB, Canada) throughout the experiment, in accordance with the guidelines of the Canadian Council of Animal Care. The room that houses the crates is fitted with the Model PEC Proportional Environment Control system (Phason, Winnipeg, MB, Canada). Ambient temperature in the room was set at 15° C. for the duration of the experiment. The steers were kept indoors from Monday to Friday and were let out into a courtyard for four hours exercise on Saturdays and Sundays.

Treatments were arranged in a time-series design in which all three steers were fed the same diet for a seven-day period. The steers were offered diets with the following fibre-to-concentrate ratios: (1) week 1: 100% chopped alfalfa hay-0 wheat-barley concentrate; (2) week 2: 80% chopped alfalfa hay-20% wheat-barley concentrate; (3) week 3: 60% chopped alfalfa hay-40% wheat-barley concentrate; (4) week 4 40% chopped alfalfa hay-60% wheat-barley concentrate, and (5) week 5 20% chopped alfalfa hay-80% wheat-barley concentrate. Ad libitum intake during week 1 was used as the basis for determining intake in subsequent weeks. In weeks 2 through 5, each steer was offered a daily allocation equivalent to its daily average intake determined during week 1. The allocation was divided into the appropriate proportions of chopped alfalfa hay and wheat-barley concentrate as determined each week. During weeks 2 to 4, wheat-barley concentrate was top-dressed onto chopped alfalfa hay, mixed thoroughly and offered to the steers at 9:00 am every day. On Monday and Friday of week 5, the steers received 1 kg of chopped alfalfa hay at 9:00 am, two meals of 3.2 kg each of wheat-barley concentrate at 11:00 am and 1:00 pm and 1 kg of chopped alfalfa hay at 5:00 pm. During the other days of the week, steers received a diet with a fibre-to-concentrate ratio of 40:60. Weigh-backs were weighed and sampled for dry matter determination. Dry matter (%), contents were 92.45% for chopped alfalfa hay and 89.24% for wheat-barley concentrate.

Rumen pH was measured continuously for two 24-hour periods during each week by inserting one indwelling pH probe into the rumen of each steer. For weeks 1 to 4, probes were inserted on Thursday at 9:00 am and were taken out on Saturday at 9:00 am. During week 5, probes were inserted on Monday at 9:00 am, taken out on Tuesday 9:00 am, and reinserted at 9:00 am on Thursday. The indwelling pH probes were placed into the ventral sac of the rumen of each steer and kept in position by means of a 0.5 kg weight as described by Cumby et al. (2001, Can. J. Anim. Sci. 81: 149-152). Rumen pH was measured every second averaged over 60 sec, and the minutely averages were captured by a data logger and stored on a diskette for further analysis. Rumen fluid pH data were summarized as average pi time below pH 6.0 and time below pH 5.6, area (time×pH),below pH 6.0, and area (time×pH) below pH 5.6 for each 24-hour period.

Rumen fluid samples were also collected into sterile plastic tubes from the ventral sac of the rumen at 9:00 am, 12:00 pm, 3:00 pm, 9:00 pm and 3:00 am during the time that rumen pH was measured and were processed for LPS determinations as described in Example 1. A second set of rumen fluid samples was collected at 9:00 am, 12:00 pm, 3:00 pm and 9:00 pm into sterile plastic vials with airtight lids and used immediately to determine total coliform counts. Portion of these samples were centrifuged at 3,000 rpm for 10 minutes and the supernatants stored at −20° C. for VFA analysis.

The Limulus amebocyte lysate (LAL) assay was used to determine LPS concentrations following the method described by Levin et al. (1964, Bull. John Hopkins Hosp. 115: 337). The assay was performed using a 96-well microplate kit (Cambrex Bio Science, Walkersville, Md., USA) with absorbance read at 405 nm using a microplate reader (Bio-Rad® model 3550, Bio-Rad Laboratories Inc., Hercules, Calif., USA). The procedures for sample preparation and method validation were followed as described in Example 1.

Volatile fatty acids were determined by thawing the previously frozen rumen fluid samples at room temperature, then adding 1 mL of a 25% meta-phosphoric acid solution to 5 mL of each rumen fluid sample. The tubes containing the mixture were vortexed and placed into a −20° C. freezer for 17 h after which, samples were thawed and centrifuged for 10 min at 1900 xg. Approximately 2 mL of supernatant were decanted from each tube into a clean dry vial which was capped and placed into the autosampler device (Model 8100; Varian, Walnut Creek, Calif.). Concentrations of VFA were determined by gas chromatography (Model 3400 Star; Varian) using a 1.83-m glass column (Model 2-1721; Supelco, Oakville, ON, Canada) as described by Erwin et al. (1961, J. Dairy Sci. 44: 1768-1776). The injector and detector temperatures were set at 170° ^{C} and 195° C., with initial and final column temperatures set at 120° C. and 165° C., respectively. The run time was 4 min followed by a 2-min thermal stabilization period.

Blood samples were collected twice via extended use polyurethane catheters (Mila Cath® , Mila International, Florence, Ky., USA) fitted into the jugular vein of each steer. Samples were collected at 9:00 am and 9:00 pm on the days that rumen pH was measured (Thursday and Friday for weeks 1 to 4; Monday and Thursday for week 5). Two 10-mL blood samples were collected into serum and plasma tubes. Serum and plasma were harvested by centrifuging samples at 3,000 rpm for 30 minutes. Haptoglobin and serum amyloid-A were determined in serum and plasma, respectively, using ELISA Tridelta® Phase range assay kits (Tridelta Diagnostics Inc, Morris Plains, N.J., USA; catalogue numbers TP-801 and TP-802, respectively). Methods for determining the acute phase proteins were followed as described in Example 1.

Chromogenic medium (E. coli/coliform medium catalogue number CM0956, Oxoid Inc. Nepean, ON, Canada) was used for culturing rumen coliform bacteria while buffered peptone water (Difco®, Becton Dickinson and Co., Mississauga, ON, Canada) was used for the serial dilutions. The chromogenic medium is normally used to differentiation between Escherichia coli and other coliforms in cultures isolated from food and environmental samples. Chromogenic medium was prepared per the manufacturer's instructions except that 20% of the purified water was replaced with clarified rumen fluid. The rumen fluid was collected from a fistulated cow fed a 60% concentrate diet. The rumen fluid was centrifuged at 3,000 rpm for 10 minutes. The supernatant was collected and autoclaved at 121° C. for 15 minutes, then stored frozen until required for making the chromogenic medium. Clarified rumen fluid was added prior to autoclaving the medium. Carbon dioxide gas was bubbled through buffered peptone water and the chromogenic E. coli/coliform medium immediately after autoclaving in order to reduce the oxygen tension.

100 μL of rumen fluid was inoculated using aseptic technique, into 900 μL of a 2% buffered peptone water (Difco®) in a 2-ml sterile deep well plate, thoroughly mixed by aspirating and dispensing the mixture several times, then serially-diluted to the appropriate dilution. Sterile pipette tips were used for each dilution. Drop-plating was performed and ten 50-μL drops were pipetted (Eppendorf Repeater® 4780, Brinkman Instruments Inc., Westbury, N.Y., USA) onto agar plates containing modified chromogenic E coli/coliform medium. After the drops on the agar dried, the plates were inverted and incubated at 37° C. for 18 h. Colonies were manually counted and broadly classified according to colour with purple being designated E coli and pink to brown colonies as other coliforms. The countable dilution was determined as the dilution that gave 3 to 30 colonies per drop of sample dispensed.

Data were analyzed using mixed model analysis with the most appropriate covariance structure as described by Wang and Goonewardene (2004, Can. J. Anim. Sci. 84: 1-11). The following covariance structures were deemed appropriated for the variables in parenthesis; first order ante dependence (average rumen pH); unstructured covariance (time<pH 5.6, min/d; time<pH 6.0, min/d; area<pH 5.6, min×pH/d; area<pH 6.0, min×pH/d); compound symmetry (total VFA, ruminal LPS, serum Hp and plasma SAA concentrations) and simple (total coliform counts) using SAS/STAT® software. The experimental unit was the steer and classification variables were steer, treatment, and day for rumen pH and total VFA or hour of sampling for acute phase proteins and LPS data. The following generalized model was used: Yᵢⱼ=μ+αᵢ+βⱼ+(α×β)ᵢⱼ+eᵢⱼ where Yᵢⱼ=observations for dependent variables, μ=overall mean; αᵢ=average effect of treatment i; βⱼ=average effect of the day or hour j; (α×β)ᵢⱼ=interaction between either treatment and day or treatment and hour and eᵢⱼ=error. LPS concentrations and total coliform counts were log-transformed to correct non-homogenous residual errors. Since treatments were separated in time, orthogonal contrasts were used to compare treatment 1 to all other treatments for all variables. However, where a diurnal variation of a variable was of interest and in the absence of a significant interaction, Tukey's multiple range test was used to separate means.

Table 3 shows that the average dry matter intake were 5.9 kg, 5.8 kg, 5.9 kg, 5.9 kg and 5.9 kg per day for treatments 1 to 5, respectively when steers were fed the wheat-barley concentrate. Dry matter intake was not affected by treatment but there was a significant day-to-day variation (P=0.04) as well as a day-by-treatment is interaction (P=0.02). The target and actual feed-to-concentrate ratios in the diet were similar. Steers consumed all the feed offered during weeks 2 to 4. During week five as much as 15% of DM intake was left over.

TABLE 3
Dry matter intake of steers offered graded levels of wheat-barley concentrate
and chopped alfalfa hay (means ± SD).

| | Week¹ | | | | |
|---|---|---|---|---|---|
| | 1 | 2 | 3 | 4 | 5 |
| Intended feed-to- | 100:0 | 80:20 | 60:40 | 40:60 | 20:80 |
| concentrate ratio | n = 21 | n = 21 | n = 21 | n = 36 | n = 6 |
| Intake (as fed) (kg/d) | | | | | |
| Chopped alfalfa hay | 6.4 ± 2.8 | 5.1 ± 0.5 | 3.9 ± 0.3 | 2.5 ± 0.5 | 1.8 ± 0.4 |
| Wheat-barley concentrate | 0 | 1.3 ± 0.1 | 2.6 ± 0.2 | 3.5 ± 1.0 | 6.0 ± 0.5 |
| Intake (DM basis)(kg/d) | | | | | |
| Chopped alfalfa hay | 5.9 ± 2.5 | 4.7 ± 0.4 | 3.6 ± 0.3 | 2.3 ± 0.4 | 1.6 ± 0.4 |
| Wheat-barley concentrate | 0 | 1.2 ± 0.1 | 2.3 ± 0.3 | 3.1 ± 0.9 | 5.3 ± 0.4 |
| Orts/d (% DM offered) | 41 | 0 | 0 | 8.5 | 8.3 |
| Total DM intake (kg/d) | 5.9 | 5.9 | 5.9 | 5.4 | 6.9 |
| Actual feed-to-concentrate | 100:0 | 80:20 | 60:40 | 42:58 | 23:77 |
| Ratio | | | | | |

¹Daily average feed intake for week 4 is the average week 4 intake and the 5 days in week 5 when steers were on the 40:60 F:C ratio and only week 5 intake is the average of two days during which SARA was induced.

Table 4 shows that the average rumen pH tended (P=0.057) to be lower for the treatment 4 but was significantly lower (P=0.005) for treatment 5 compared to treatment 1. Similarly, the time the steers spent with pH below 6 (P=0.07) tended to be greater than treatment, 4 but was significantly higher (P=0.003) in treatment 5 compared to treatment 1. Time with pH below 5.6 was only significantly greater in treatment 5 compared to treatment 1. Time area under the pH×time curve for both the 5.6 and 6.0 pH thresholds tended (P<0.1) to be higher only during treatment 5 compared to treatment 1.

TABLE 4
Rumen pH variables of steers offered graded levels of wheat-barley
concentrate and chopped alfalfa hay.¹

| | Week | | | | | | | | |
|---|---|---|---|---|---|---|---|---|---|
| | 1 | 2 | 3 | 4 | 5 | | | | |
| | Intended F:C ratio | | | | | | P value | | |
| | 100:0 | 80:20 | 60:40 | 40:60 | 20:80 | SEM | Trt | Day | Diet × Trt |
| Average pH | 6.72^{a} | 6.69^{ab} | 6.64^{ab} | 6.44^{b} | 6.14^{c} | 0.17 | 0.003 | 0.41 | 0.88 |
| Time < pH 5.6 min/d | 0^{b} | 0^{b} | 0^{b} | 121^{ab} | 219^{a} | 61.6 | 0.069 | 0.71 | 0.99 |
| Time < pH 6.0 min/d | 22^{bc} | 0^{c} | 10^{bc} | 344^{ab} | 600^{a} | 119.3 | 0.007 | 0.78 | 0.30 |
| Area < pH 5.6, min × pH/d | 0 | 0 | 0 | 17 | 38 | 15.2 | 0.34 | 0.23 | 0.68 |
| Area < pH 6.0, min × pH/d | 1 | 0 | 1 | 101 | 216 | 73.4 | 0.20 | 0.38 | 0.65 |

¹Orthogonal contrasts were used to separate means to determine significant treatment effects.
^{a,b,c}Means with different superscripts within each row were significantly different (P ≦ 0.05).

TABLE 5
Haptoglobin (Hp) and Serum amyloid-A (SAA) concentrations in
steers fed diets with different feed-to-concentrate ratios.¹

| | Week | | | | | | | | |
|---|---|---|---|---|---|---|---|---|---|
| | 1 | 2 | 3 | 4 | 5 | | | | |
| | Intended F:C ratio | | | | | | P value | | |
| | 100:0 | 80:20 | 60:40 | 40:60 | 20:80 | SEM | Trt | Day | Diet × Trt |
| Hp (mg/mL) | 0.53^{b} | 0.54^{b} | 0.56^{b} | 1.90^{a} | 1.40^{a} | 0.23 | 0.0008 | 0.78 | 0.99 |
| SAA (μg/mL) | 38^{b} | 49^{b} | 55^{b} | 71^{b} | 163^{a} | 13.83 | <0.0001 | 0.23 | 0.44 |

¹Orthogonal contrasts were used to separate means to determine significant treatment effects.
^{a,b,c}Means with different superscripts within each row were significantly different (P ≦ 0.05).

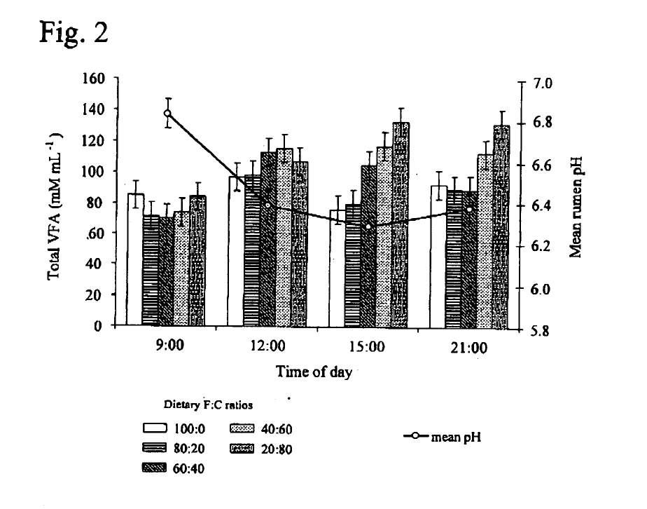

Total volatile fatty acids were measured to indicate changes in rumen pH. The concentration of VFA was influenced by both treatment and time after feeding (FIG. 2). Concentrations increased to peak around 12 noon for treatments 1 to 3, but peaked around 3:00 pm for treatments 4 and 5.

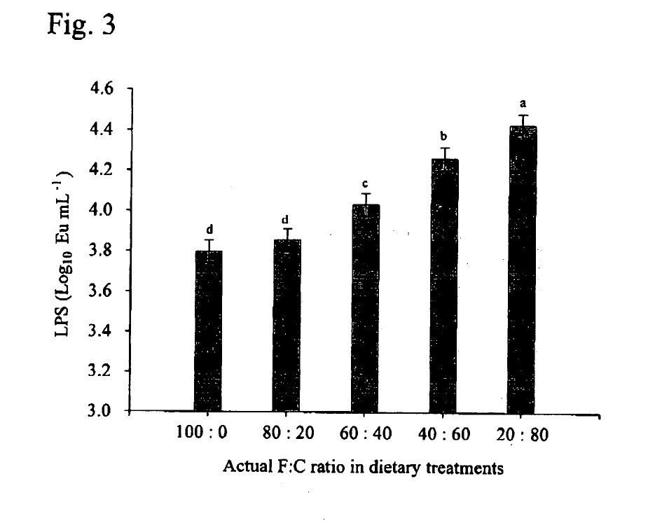

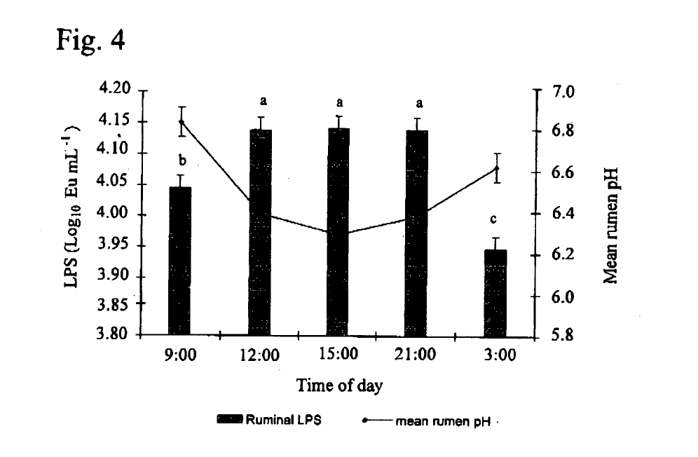

Ruminal LPS concentration were significantly (P<0.0001) different among the treatments (FIG. 3). LPS concentrations increased with increasing level of graded wheat-barley concentrate (FIG. 3). There was a significant (P<0.0001) diurnal variation in LPS concentration (FIG. 4). In the absence of a treatment×hour interaction data were pooled across treatments to present diurnal variations. LPS increased from 4.039 at 9:00 am to a peak of 4.135 Log₁₀ Eu mL⁻¹ at 3:00 pm (FIG. 4).

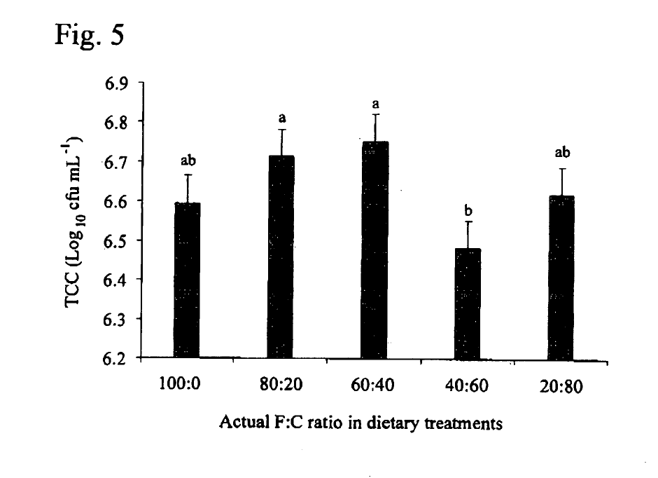

In the absence of any other general group that could be used to determine changes in ruminal LPS concentration, total coliform counts were used to determine trends in Gram-negative bacteria numbers as the rumen pH changed. Total coliform counts for samples collected at 9:00 am during weeks 1 to 5 were used to determine the effects on coliforms of adding graded levels of wheat-barley concentrate total mixed rations. The samples that were collected during the four time intervals during weeks 2 to 5 were used to determine diurnal variations in total coliform counts. Thus, using only the samples collected at 9:00 am, total coliform concentrations were 6.60, 6.71, 6.75, 6.48 and 6.62 log₁₀ CFU mL⁻¹ in rumen fluid (FIG. 5). When comparing the 100:0 feed-to-concentrate ratio treatment to each of the other treatments, there were no differences (P>0.10) in total coliform counts in the rumen fluid during the times that the diets with different feed-to-concentrate ratios were fed except in treatment 3. There were no significant diurnal variations (P>0.10) in total coliform concentrations in samples collected during the four time intervals during weeks 2 to 5 (data not shown).

Serum haptoglobin concentrations were affected by dietary treatments with fibre-to-concentrate ratios of 40:60 and 20:80 (Table 5). The haptoglobin concentrations increased from 0.53 mg mL⁻¹ in steers fed the basal diet to 1.90 mg mL⁻¹ in steers on the diet with fibre-to-concentrate ratio of 40:60. Plasma concentrations of serum amyloid-A were also affected by dietary treatment (Table 5). Concentrations tended (P=0.09) to be higher in steers fed 40:60 fibre-to-concentrate diet compared to the basal diet and were significantly (P<0.0001) higher in steers on the 20:80 fibre-to-concentrate ratio diet.

Serum haptoglobin concentrations were elevated (P<0.05) with both the 40:60 and 20:80 fibre-to-concentrate diets. Plasma serum amyloid-A concentrations were also affected by diets with lower fibre-to-concentrate ratios. These diets typically resulted in animals experiencing varying degrees of SARA. The response in serum amyloid-A was lower during the time that the 40:60 fibre-to-concentrate diet. The serum amyloid-A concentrations obtained with the 20:80 fibre-to-concentrate diet was comparable to those obtained in Example 1 during the latter stages when SARA was induced for 5-day periods at a time. Acute phase proteins are released from hepatocytes upon stimulation by inflammation mediators which include interleukin-1, interleukin-6 and tumor necrosis actor. The quantification of acute phase proteins, i.e., serum amyloid-A and haptoglobin, in this example and in Example 1 indicates that SARA initiates an inflammatory response.

In this experiment, gradual stepwise adaptation to a wheat-barley concentrate affected ruminal LPS concentration. LPS concentration increased with increasing concentrate in the diet. The number of coliform bacteria that was used to indicate changes in gram-negative bacteria, initially increased but concentrate inclusion that induced SARA also showed no change in coliform numbers when compared to the all forage diet. The concentration of the two acute phase proteins used as inflammation markers in this study were increased when concentrate inclusion induced SARA. These results data show the importance of acute phase proteins in the early detection of SARA in the absence of overt signs.

### EXAMPLE 3

Four primiparous, ruminally fistulated Holstein dairy cows were housed in individual tie stalls in the Metabolism Unit of the Glenlea Dairy Research Farm in Winnipeg, MB Canada, in accordance with the guidelines of the Canadian Council of Animal Care (1993). Test animals were lactating cows with an average body weight (BW) of 538±12.4 kg, that were 121±8 (mean±SD) “days in milk” (DIM) with an average milk yield of 30±4.3 (mean±SD) kg d⁻¹ at the beginning of the experiment. The experiment was conducted as a switchover design with four periods of 1 wk. During each time period, SARA was induced in two cows by replacing 25% of the dry matter intake of total mixed rations measured in the previous period with an equal amount of dry matter of wheat-barley concentrate. The amount of wheat-barley concentrate fed to each cow averaged 4.4±0.4 (mean±SD) kg d⁻¹ and was offered in two daily meals with 67% of the daily allocation offered at 11:00 am and the remaining 33% at 2:00 pm. The total mixed rations was withheld between 11:00 am and 5:00 pm. The other two cows received a total mixed rations diet only (i.e., control). The diet treatments were switched each subsequent week. Cows in both groups had ad libitum access to fresh water. The ingredients and chemical composition of the total mixed rations and wheat-barley concentrate are shown in Table 6. Feed intakes were determined daily, and representative feed and orts samples were taken three times per week. Samples were pooled by week, and feed intakes and dry matter contents were determined by oven drying at 60° C. for 48 h.

Rumen pH was measured continuously for 5 days in each treatment period by inserting one in-dwelling pH probe into the rumen of each cow as described in Example 1. Rumen pH was measured at 1-min intervals and send to a data logger for storage and subsequent analysis. Rumen pH data were summarized by calculating average pH, time below pH 6, and time below pH 5.6 area (time×pH below pH 6 and area below pH 5.6) for each 24-h period. Time with rumen pH below 6.0 was monitored because microbial fibre digestion and nutrient digestibilities are reduced below this rumen pH level. Time with rumen pH below 5.6 was also used because this defines the upper limit of the range of rumen pH that defines SARA. Rumen fluid samples were collected into sterile plastic tubes from the ventral sac of the rumen at 3:00 pm from Monday to Friday. Samples were also collected at 6-h intervals starting at 9:00 on Wednesday during each week.

TABLE 6
Feed ingredients and composition for: (a) total
mixed rations, and (b) wheat-barley concentrate.

| | Total mixed | Wheat-barley |
|---|---|---|
| Component | rations | concentrate |
| Ingredients, % | | |
| Alfalfa silage | 32.6 | |
| Oat silage | 22.35 | |
| Energy supplement¹ | 35.24 | |
| Protein supplement² | 8.81 | |
| Ground wheat | | 50.0 |
| Ground barley | | 50.0 |
| Nutrient composition | | |
| Dry matter (DM), % | 63.39 | 89.2 |
| Crude protein, % DM | 18.5 | 15.5 |
| Neutral detergent fibre, % DM | 31.1 | 17.6 |
| ADF | 23.3 | 5.9 |
| Non-structural carbohydrates, % DM | 37.2 | 62.2 |
| Calcium, % DM | 1.25 | 0.27 |
| Phosphorus, % DM | 0.53 | 0.35 |
| Potassium, % DM | 1.72 | 0.56 |
| Mg, % DM | 0.38 | 0.16 |
| Na, % DM | 0.36 | 0.03 |

¹Energy supplement contained 0.13% APE premix, 0.14% trace mineral premix, 2.6% soybean meal, 0.06% selenium, 39.1% wheat shorts, 5.0% distillers grain, 17.5% canola meal, 15.0% ground wheat, 1.7% dicalcium phosphate, 1.6% salt, 2.0% dynamite, 0.3% pellet binder, 1.0% cane molasses, 3.7% calcium carbonate, and 10.0% corn gluten meal.
²Protein concentrate contained 46.1% soybean meal, 2.6% wheat shorts, 40.0% canola meal, 5.0% oat hulls, 0.3% pellet binder, 1.0% cane molasses, and 5.0% corn gluten meal.

A 25-mL portion of each sample of rumen fluid was processed for subsequent free ruminal LPS determination as described in Example 1. The Limulus amebocyte lysate (LAL) assay was used to determine LPS concentration. The assay was performed using a 96-well microplate kit (Cambrex Bio Science Inc., Walkersville, Md., USA) with absorbance read at 405 nm on a microplate reader (Bio-Rad® model 3550, Bio-Rad Laboratories Inc., Hercules, Calif., USA).

A second 1.5-mL portion of rumen fluid was mixed with an equal volume of glycerin in plastic tubes and stored at −20° C. until used for total coliform counts at a later stage. Total coliform counts were determined as described in Example 2.

The remaining portion of rumen fluid was centrifuged at 1500 xg for 10 minutes and the supernatant stored at −20° C. for VFA analysis. A day before analysis, rumen fluid samples were thawed at room temperature and 1 mL of a 25% meta-phosphoric acid solution was added to 5 mL of rumen fluid. The tubes containing the mixture were vortexed and placed in a −20° C. freezer for 17 h after which, samples were thawed and centrifuged for 10 min at 1500 xg. Approximately 2 mL of supernatant were decanted into a clean dry vial which was capped and placed into the auto-sampler device (Model 8100; Varian Inc., Walnut Creek, Calif., USA). VFA concentrations were determined by gas chromatography as described in Example 2.

Blood samples were collected into four blood tubes for each cow at 9:00 am and 3:00 pm on Wednesday during each time period. Four samples were collected from each cow into: two 10-mL serum tubes (plain), one 10-mL green top plasma tube (sodium heparin coated) and one 5-mL purple top (K₃EDTA coated) Vacutaimer® tubes (Becton Dickinson and Co., Franklin Lakes, N.J., USA). Plasma fibrinogen was determined by refractometry according to the method described by George (2001, Vet. Clin. Pathol. 30: 201-210). Copper in serum was analyzed by atomic absorption spectrophotometer (Varian AA240FS, Varian Inc., Walnut Creek, Calif., USA). An automated hematology analyzer (Cell-Dyn® 3500 system, Abbott Laboratories, Abbott Park, Ill., USA) was used for enumeration and delineation of white blood cells in whole blood. White blood cells were differentiated into neutrophils, eosinophils, lymphocytes, monocytes, basophils, and bands where present. Thrombocyte counts were done using the manual method of Brecher et al. (1950, J. Appl. Physiol. 3: 365-377). All analyses were done at the Manitoba Veterinary Services Laboratory (Winnipeg, MB, Canada) in blood serum or K₃EDTA-preserved whole blood.

The second sample in the serum tube was left to clot at room temperature, which was about 18° C., for 45 min and then centrifuged at 1500 xg for 30 min. The sodium heparin-preserved blood sample was centrifuged at 1500 xg immediately after collection. Serum and plasma were harvested and stored at −20° C. Haptoglobin and serum amyloid-A were determined in serum and plasma, respectively, using ELISA Tridelta® Phase range assay kits as described in Example 1.

Serum was also used to determine blood concentration of LPS using the Limulus amebocyte lysate (LAL) assay and the same method as in ruminal LPS analyses except that Pyrosperse® (Cambrex Bio Science Walkersville Inc., MD), a metallo-modified polyanionic dispersant was added to the samples prior to analysis. Pyrosperse® is recommended for use with serum to prevent endotoxin binding. Serum samples were diluted 1:5 with pyrogen-free water and the assay was also performed using a 96-well microplate kit with absorbance read at 405 nm on a microplate reader as previously described.

Cows were milked twice daily in their stalls, and milk production was determined using Tru Test® regulation meters (Westfalia Surge, Mississauga, ON, Canada). Milk samples were collected from 4 consecutive milkings in 50-mL vials in each collection period and preserved with 2-bromo-2-nitropropane-1,3 diol. Milk samples were collected from Tuesday afternoon milking through to Thursday morning milling and stored at 4° C. until analyzed at the laboratory of the Dairy Farmers of Manitoba (Winnipeg, MB, Canada) by near-infrared analysis for milk fat and milk protein content using the Milk-O-Scan 303AB (Foss Electric, Hillerad, Denmark) and for somatic cell counts using the Fossomatic® 300 cell counter (Foss Electric, Hillerad, Denmark).

Data were analyzed using SAS Mixed Procedures from SAS/SYSTAT as recommended by Wang and Goonewardene (2004, Can. J. Anim. Sci. 84: 1-11) for the analysis of animal experiments with repeated measures. The effects of diet, time (either hour or day), and their interactions were considered fixed. Week effects were considered random. Compound symmetry was deemed the most appropriate covariance structure relative to the hourly or daily measurements that were tested. Tukey's multiple range test was used to separate means after a significant (P<0.05) treatment or time effect.

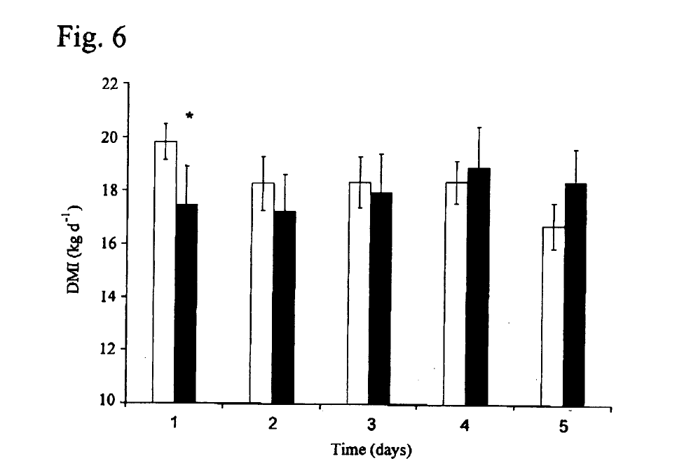

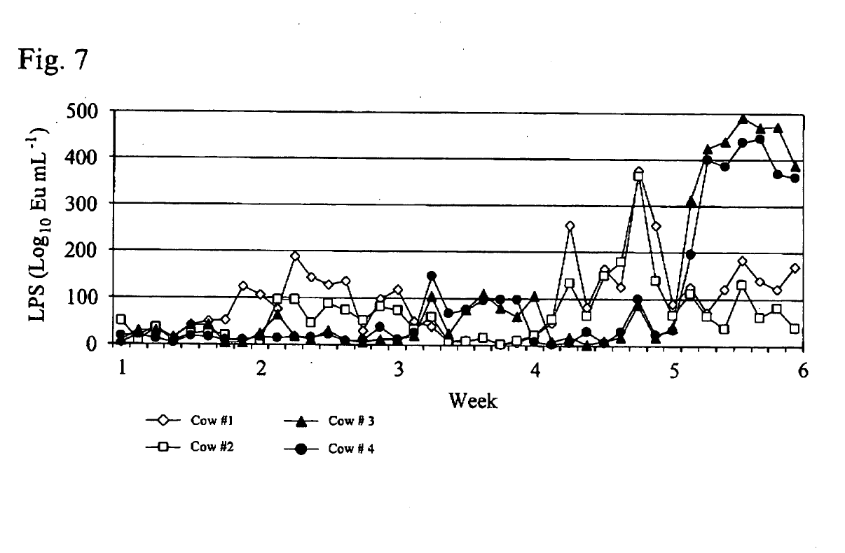

Total dry matter intake did not differ between treatments and averaged 18.0 and 18.3 kg cow⁻¹ for the control and SARA treatments, respectively (Table 7). On average, total dry matter intake was higher (P=0.03) on the first day of SARA induction but was numerically lower (P=14) on the last day of SARA induction for cows receiving the SARA diet compared to Cows receiving total mixed rations (FIG. 6). This resulted in a treatment×day interaction (P=0.003) for both total mixed rations and total dry matter intake (Table 7).

Somatic cell counts (SCC) were used to screen cows with subclinical mastitis, as this disease can also result in an inflammatory response. One cow had an abnormally high SCC for the first 3 weeks of the study which averaged 991,542 with a range of 16,000 to 4,087,000 cells mL⁻¹. Data on inflammation markers in blood samples from this cow were excluded from the analyses. The SCC for the other three cows averaged 98,125 cells mL⁻¹ with a range of 21,000 to 299,000 cells mL⁻¹. Milk yields and milk composition are shown in Table 7. Milk yield, fat yield and protein yield did not differ between SARA and control cows. Milk at content did not differ between SARA and control cows (Table 7).

TABLE 7
Effect of diet of dry matter intake, milk yield and composition.

| | | | | P value | | |
|---|---|---|---|---|---|---|
| | Diet | | | | | Diet × |
| | Control² | SARA³ | SEM | Diet | Day | Day |
| Item¹ | | | | | | |
| TMR DMI (kg/d) | 18.0^{a} | 14.0^{b} | 1.66 | <0.0001 | 0.12 | 0.003 |
| Total DMI (kg/d) | 18.0 | 18.3 | 1.64 | 0.74 | 0.04 | 0.002 |
| Milk yield (kg/d) | 27.5 | 28.6 | 1.83 | 0.27 | 0.81 | 0.69 |
| Milk components | | | | | | |
| Milk fat (%) | 2.49 | 2.21 | 0.23 | 0.45 | 0.70 | 0.08 |
| Fat yield (kg/d) | 0.68 | 0.63 | 0.04 | 0.43 | 0.94 | 0.91 |
| Milk protein (%) | 3.60 | 3.39 | 0.14 | 0.30 | 0.78 | 0.13 |
| Milk protein | 1.01 | 0.98 | 0.08 | 0.77 | 0.86 | 0.76 |
| (kg/d) | | | | | | |

¹Dry matter intakes (DMI) were calculated for 2 animals for each treatment across the time periods (n = 40), and for 4 animlas averaged across periods and treatments for “day” effects (n = 16); milk yield and composition were from samples collected on 2 days during each week (n = 12) for treatment and day effects (n = 4) and analyzed by analysis of variance.
²Control diet was total mixed ration (TMR).
³SARA diet was TMR plus 25% wheat-barley concentrate.
^{a,b}means within the same row followed by different superscript letters were significantly different (P ≦ 0.05)

Inducing SARA caused a significant decrease (P=0.0008) of 0.23 units in average daily rumen pH in cows fed the SARA diet compared to cows fed total mixed rations (Table 8) and increased (P=0.0008) the duration of time with pH below 6.0 from 459.5 min d⁻¹ to 741.9 min d⁻¹ and also increased (P<0.0001) the duration of time with pH below 5.6 from 187.0 min d⁻¹ to 309.4 min d⁻¹. The area (time×pH) below pH 6.0 increased (P<0.0001) from 145.7 to 278.7 and the area (time×pH) below pH 5.6 increased (P=0.001) from 28.4 to 67.0 when SARA was induced (Table 8).

TABLE 8
Effects of diet on rumen pH.

| | Diet | | | P value | | |
|---|---|---|---|---|---|---|
| Item¹ | TMR² | SARA³ | SEM | Diet | Day | Diet × Day |
| Average pH | 6.24^{a} | 6.01^{b} | 0.065 | 0.0008 | 0.75 | 0.69 |
| Time < pH 5.6 min/d | 187.0^{b} | 309.4^{a} | 32.1 | <0.0001 | 0.59 | 0.99 |
| Time < pH 6.0 min/d | 459.5^{b} | 741.9^{a} | 61.5 | 0.0008 | 0.74 | 0.87 |
| Area < pH 5.6, min × pH/d | 28.4^{b} | 67.0^{a} | 9.7 | 0.001 | 0.12 | 0.95 |
| Area < pH 6.0, min × pH/d | 145.7^{b} | 278.7^{a} | 24.8 | <0.0001 | 0.48 | 0.98 |

¹Dry matter intakes (DMI) were calculated for 2 animals for each treatment across the time periods (n = 40), and for 4 animlas averaged across periods and treatments for “day” effects (n = 16); milk yield and composition were from samples collected on 2 days during each week (n = 12) for treatment and day effects (n = 4) and analyzed by analysis of variance.
²Control diet was total mixed ration (TMR).
³SARA diet was TMR plus 25% wheat-barley concentrate.
^{a,b}means within the same row followed by different superscript letters were significantly different (P ≦ 0.05)

Total VFA concentrations in the rumen were not affected by treatment and were 137.2 mML⁻¹ and 129.7 mML⁻¹ for SARA and control cows, respectively (Table 9). Ruminal propionic acid concentration increased (P<0.0001) from 26.1 mML⁻¹ in cows fed total mixed rations to 39.81 mML⁻¹ in cows fed the Sara diet. The SARA diet also caused a lower Ac/Pr ratio (P<0.0001) (Table 9). Averaged across time periods and treatments, highest propionic and butyric acid concentrations were measured at 9:00 pm (Table 9).

TABLE 9
Effects of diet¹ on volatile fatty acid concentrations (mM/L)
in rumen fluid².

| | | | | | | Ac/PR |
|---|---|---|---|---|---|---|
| Variable | Acetate | Propionate | Butyrate | Other³ | Total | ratio⁴ |
| Time of | | | | | | |
| day | | | | | | |
| 9 am | 84.5 | 27.3^{b} | 8.5^{c} | 4.0^{a} | 124.4 | 3.3^{a} |
| 3 pm | 84.5 | 34.8^{a} | 11.0^{ab} | 3.5^{ab} | 133.8 | 2.7^{b} |
| 9 pm | 91.2 | 35.8^{a} | 12.6^{a} | 3.3^{b} | 142.9 | 2.7^{b} |
| 3 am | 85.5 | 33.9^{a} | 9.8^{bc} | 3.5^{ab} | 132.7 | 2.7^{b} |
| SEM | 6.76 | 3.53 | 1.12 | 0.36 | 10.8 | 0.18 |
| Diet⁵ | | | | | | |
| Control | 88.9 | 26.1^{b} | 10.9 | 4.0^{a} | 129.7 | 3.5^{a} |
| SARA | 84.0 | 39.8^{a} | 10.1 | 3.2^{b} | 137.2 | 2.2^{b} |
| SEM | 6.22 | 3.30 | 1.11 | 0.31 | 9.84 | 0.20 |

¹Diets: (a) Control was TMR, (b) SARA was TMR plus 25% wheat-barley concentrate.
²Rumn fluid volatile fatty acid concentrations were averaged for 2 animals across periods (n = 16) for each sampling time, across sampling times, and across periods (n = 32) for each treatment, and were analyzed by analysis of variance.
³Other acids were isobutyric acid, isovaleric acid, and valeric acid.
⁴Acetate:Propionate ratio
⁵Dietary means were calculated across sampling times.
^{a,b,c}Means for variables in the same column followed by different superscript letters were significantly different (P ≦ 0.05).

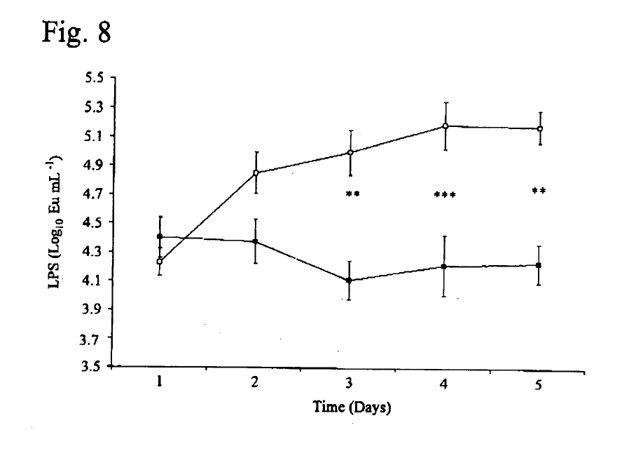

Inducing SARA increased (P<0.0001) free ruminal LPS 22,908 to 128,825 Eu mL⁻¹ across the sampling times (Table 10). There was a diurnal variation in free ruminal LPS, and averaged across treatments, the lowest concentrations were measured in samples collected at 3:00 pm. For the entire period of the study, free ruminal LPS concentrations were relatively higher during the second SARA challenge compared to the first (FIG. 7). In order to determine the day-to-day variation, free ruminal LPS was measured in samples collected at 3:00 pm daily. The difference in free ruminal LPS between cows fed the control and SARA diets, progressively increased up to the fourth day of treatment (FIG. 8). Serum LPS concentrations were below the LAL assay detection limit of 0.1 Eu mL⁻¹.

TABLE 10
Effects of diet on ruminal LPS, rectal temperature, serum amyloid-A,
haptoglobin and other blood chemistry variables.

| | Diet² | | | P value | | |
|---|---|---|---|---|---|---|
| | Control | SARA | SEM | Diet | Day | Diet × Day |
| Item¹ | | | | | | |
| Ruminal LPS (log₁₀Eu/ml) | 4.39^{b} | 5.11^{a} | 0.15 | <0.0001 | <0.0001 | 0.43 |
| Rectal temperature (° C.) | 38.4 | 38.5 | 0.20 | 0.73 | 0.004 | 0.20 |
| Serum amyloid-A (μg/mL) | 268.8^{b} | 498.8^{a} | 85.6 | 0.03 | 0.61 | 0.68 |
| Haptoglobin (mg/mL) | 0.244 | 0.265 | 0.03 | 0.59 | 0.17 | 0.17 |
| Fibrinogen (mg/mL) | 4.50 | 5.00 | 0.29 | 0.24 | 0.99 | 0.40 |
| White blood cells | | | | | | |
| Neutrophils (%) | 43.5 | 44.8 | 2.89 | 0.60 | 0.41 | 0.85 |
| Eosinophils (%) | 3.20 | 2.29 | 0.39 | 0.10 | 0.53 | 0.90 |
| Lymphocytes (%) | 46.10 | 45.49 | 2.61 | 0.57 | 0.65 | 0.91 |
| Monocytes (%) | 6.50 | 6.08 | 1.01 | 0.68 | 0.46 | 0.93 |
| Basophils (%) | 0.75 | 0.94 | 0.23 | 0.53 | 0.61 | 0.09 |
| Bands³ (%) | 0.28 | 0.32 | 0.22 | 0.91 | 0.59 | 0.69 |
| Thrombocytes | 476.00 | 466.17 | 68.8 | 0.92 | 0.51 | 0.21 |

¹Ruminal LPS concentrations and rectal temperatures were calculated for the animals on each treatment averaged across periods and times of sampling (n = 32) and for time intervals (n = 16); blood chemistry variables (n = 16) for treatment effects and time intervals (n = 32), and where analyzed by analysis of variance.
²Diets: (a) Control was TMR, (b) SARA was TMR plus 25% wheat-barley concentrate.
³Bands = immature white blood cells.
^{a,b}Means for variables within the same row followed by different superscript letters were significantly different (P ≦ 0.05).

Rectal temperatures did not differ between treatments and were 38.4° C. and 38.5° C. for cows fed control and SARA diets, respectively (Table 10). Averaged across period and treatment, lowest rectal temperatures were recorded at 3:00 pm within a 24 hour period (P=0.004) (Table 10). Haptoglobin, fibrinogen and thrombocyte counts did not differ between treatments and respective concentrations for haptoglobin, fibrinogen and thrombocyte counts were 0.24 mg mL⁻¹ and 0.27 mg mL⁻¹, 4.5 mg mL⁻¹ and 5.0 mg mL⁻¹, and 476.0 mg mL⁻¹ and 466.2 mg mL⁻¹, for cows fed control and SARA diets, respectively (Table 10). The proportions of white blood cells did not differ between treatments and were within the normal range for cows as defined in The Merck Veterinary Manual, 9^{th} Ed. (2005, C.M. Kahn, Ed., Merck & Co. Inc.). Serum copper concentrations did not differ between treatments but inducing SARA tended (P=0.07) to depress concentration by 0.04 mg mL⁻¹. Serum amyloid-A increased (P=0.03) from 286.8 μg mL⁻¹ to 498.8 μg mL⁻¹ when SARA was induced (Table 10).

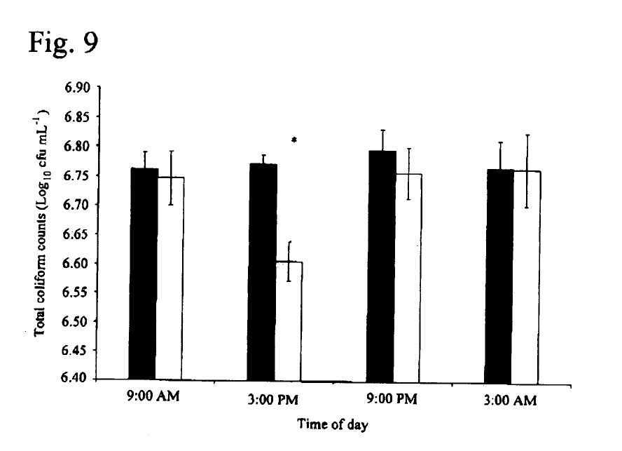

Averaged across time periods and cows within treatment, total coliform counts did not differ between treatments and were 6.77 log₁₀ cfu mL⁻¹ and 6.72 log₁₀ cfu mL⁻¹ for control and SARA cows, respectively. However, total coliform counts were lower (P=0.05) in SARA cows than in control cows for samples collected at 3:00 pm (FIG. 9).

While this invention has been described with respect to the preferred embodiments, it is to be understood that various alterations and modifications can be made to the methods and to the configuration of the diagnostic kits disclosed herein for detection and monitoring of sub-acute ruminal acidosis in livestock animals within the scope of this invention, which are limited only by the scope of the appended claims.

What is claimed is:

1. A method for detecting sub-acute ruminal acidosis in livestock animals, said method comprising:
collecting a blood sample from a livestock animal;
contacting said blood sample with a diagnostic device provided with a marker derived from at least one acute phase protein associated with inflammatory responses, the marker configured to react with said acute phase protein; and detecting a reaction between said blood sample and said marker.

2. A method according to claim 1, wherein said marker comprises an antibody configured to react with at least one acute phase protein associated with inflammatory responses.

3. A method according to claim 2, wherein said antibody is configured to react with an acute phase protein selected from the group comprising serum amyloid-A and haptoglobin.

4. A method according to claim 1, wherein said diagnostic device is provided with a plurality of markers derived from at least two acute phase proteins associated with inflammatory responses.

5. A method according to claim 4, wherein said plurality of markers comprises at least two antibodies, each of said antibodies configured to react with a selected acute phase protein associated with inflammatory responses.

6. A method according to claim 5, wherein said plurality of markers comprises at least one antibody configured to react with serum amyloid-A and at least one antibody configured to react with haptoglobin.

7. A method according to claim 1, wherein said blood sample is separated into at least a serum portion and a plasma portion.

8. A method according to claim 7 wherein said serum portion is contacted with a diagnostic device provided with a marker derived from at least one acute phase protein associated with inflammatory responses and detecting a reaction between said serum portion and said marker.

9. A method according to claim 8, wherein said marker comprises an antibody configured to react with at least one acute phase protein associated with inflammatory responses.

10. A method according to claim 9, wherein said antibody is configured to react with haptoglobin.

11. A method according to claim 7, wherein said plasma portion is contacted with a diagnostic device provided with a marker derived from at least one acute phase protein associated with inflammatory responses and detecting a reaction between said serum portion and said marker.

12. A method according to claim 11, wherein said marker comprises an antibody configured to react with at least one acute phase protein associated with inflammatory responses.

13. A method according to claim 12, wherein said antibody is configured to react with serum amyloid-A.

14. A method according to claim 8, wherein said diagnostic device is provided with a plurality of markers derived from at least two acute phase proteins associated with inflammatory responses.

15. A method according to claim 14, wherein said plurality of markers comprises at least two antibodies, each of said antibodies configured to react with a selected acute phase protein associated with inflammatory responses.

16. A method according to claim 15, wherein said plurality of markers comprises at least one antibody configured to react with serum amyloid-A and at least one antibody configured to react with haptoglobin.

17. A method according to claim 11, wherein said diagnostic device is provided with a plurality of markers derived from at least two acute phase proteins associated with inflammatory responses.

18. A method according to claim 17, wherein said plurality of markers comprises at least two antibodies, each of said antibodies configured to react with a selected acute phase protein associated with inflammatory responses.

19. A method according to claim 18, wherein said plurality of markers comprises at least one antibody configured to react with serum amyloid-A and at least one antibody configured to react with haptoglobin.

20. A method according to claim 1, wherein said blood sample is a periphery blood sample.

21. A method according to claim 1, wherein said reaction between the blood sample and the markcr is quantifiable.

22. A method according to claim 1, wherein said diagnostic device is an ELISA test kit.

23. A method according to claim 8, wherein said diagnostic device is an ELISA test kit.

24. A method according to claim 11, wherein said diagnostic device is an ELISA test kit.

25. A method according to claim 1, wherein said diagnostic device is a paper strip configured to change colour when said blood sample reacts with said marker.

26. A method according to claim 8, wherein said diagnostic device is a paper strip configured to change colour when said serum portion reacts with said marker.

27. A method according to claim 11, wherein said diagnostic device is a paper strip configured to change colour when said plasma portion reacts with said marker.

28. A diagnostic kit for detecting sub-acute ruminal acidosis in livestock animals, the diagnostic kit comprising:
the marker of claim 1; and
a solid support structure, said solid support structure engaging and cooperating therewith the marker of claim 1.

29. The diagnostic kit of claim 28, wherein said marker is an antibody configured to react with an acute phase protein selected from the group comprising serum amyloid-A and haptoglobin.

30. The diagnostic kit of claim 28, wherein said solid support structure is a paper strip.

31. The diagnostic kit of claim 28, wherein said solid support structure is an ELISA plate.

32. The diagnostic kit of claim 28, wherein said kit further comprises reactants for contacting and reacting said marker with said acute phase protein.

33. A diagnostic kit for detecting and monitoring sub-acute ruminal acidosis in livestock animals, the diagnostic kit comprising:
the plurality of markers of claim 4; and
a solid support structure, said solid support structure engaging thereon and cooperating therewith the plurality of markers of claim 4.

34. The diagnostic kit of claim 33, wherein said plurality of markers comprises at least one antibody configured to react with serum amyloid-A and at least one antibody configured to react with haptoglobin.

35. The diagnostic kit of claim 33, wherein said solid support structure is a paper strip.

36. The diagnostic kit of claim 33, wherein said solid support structure is an ELISA plate.

37. The diagnostic kit of claim 32, wherein said kit further comprises reactants for contacting and reacting said pluralities of markers with at least serum amyloid-A and haptoglobin.
